(19) United States
(12) Patent Application Publication
(10) Pub. No.: US 2018/0214570 A1
(43) Pub. Date: Aug. 2, 2018

(54) GEL COMPOSITION AND METHOD FOR PRODUCING GEL COMPOSITION

(71) Applicant: Shimadzu Corporation, Kyoto-shi (JP)

(72) Inventors: Eiichi OZEKI, Kyoto-shi (JP); Hayato MATSUI, Kyoto-shi (JP)

(73) Assignee: Shimadzu Corporation, Kyoto-shi (JP)

(21) Appl. No.: 15/747,576

(22) PCT Filed: Jan. 7, 2016
(86) PCT No.: PCT/JP2016/050407

§ 371 Date: Jan. 25, 2018

(30) Foreign Application Priority Data
Jul. 28, 2015 (JP) 2015-148362

(51) Int. Cl. A61K 47/69; A61K 47/34; A61K 47/10; A61K 9/06; B01J 13/00

(52) U.S. Cl. CPC A61K 47/6903; A61K 47/34; B01J 13/0091; A61K 9/06; A61K 47/10

(57) ABSTRACT

The gel composition according to the present invention comprises an amphiphilic block polymer that comprises a hydrophilic block chain having 20 or more sarcosine units and a hydrophobic block chain having 10 or more lactate units. The gel composition of the present invention has an excellent sustained releasability of a water-soluble drug, etc. and places a reduced burden on the living body. The gel composition may be provided in the form of an organogel, hydrogel or xerogel. A xerogel can be obtained by removing a dispersion medium from an organogel. A hydrogel can be obtained by wetting a xerogel with water or an aqueous solution.

### FIELD OF TECHNOLOGY

The present invention concerns a gel composition suitable for use as a sustained release preparation, and a manufacturing method therefor.

### BACKGROUND TECHNOLOGY

In pharmaceuticals, foods, and various other industrial fields there is a need for slow-release technology by which effective ingredients are released slowly. For example, in the administration of drugs into the body, slowing down the release of the drug from the preparation can keep the concentration of the drug in the body constant over a long period of time, reducing the number of the times the drug needs to be administered. As sustained release technology, many proposals have been made for technology that makes use of biodegradable polymers.

For example, patent reference 1 and patent reference 2 disclose technology whereby the active substance is put inside micelles of amphipathic block polymers that have hydrophilic blocks and hydrophobic blocks. With micelles of an amphipathic block polymer, the active substance can be put inside a hydrophobic core formed by hydrophobic blocks. But this technology is unsuitable for slowing the release of hydrophilic substances such as water-soluble drugs.

One known form of sustained release preparations is a solid implant that includes a drug or the like in a biodegradable-polymer matrix. For example, patent reference 3 discloses a method for subcutaneously injecting an implant precursor composition in which a lactic acid-glycolic acid copolymer (PLGA) is dissolved in a water-soluble solvent such as N-methyl pyrrolidone. With this method, when the precursor is introduced into the body, the water-soluble solvent in which the polymer is dissolved is replaced by water in the body, and the polymer is solidified by the water, thereby making it possible to form in situ in the body a depot preparation that has the property of releasing a drug slowly.

And patent reference 4 discloses that a gel composition that has the property of releasing a drug slowly can be obtained by dissolving PLGA in a mixed solvent of a non-water-soluble solvent such as ethyl benzoate and a water-soluble solvent such as N-methyl pyrrolidone.

### PRIOR ART REFERENCES

### Patent References

Patent reference 1: WO 96/20698 pamphlet

Patent reference 2: WO 2009/148121 pamphlet

Patent reference 3: WO 90/3768 pamphlet

Patent reference 4: WO 98/27963 pamphlet

### Overview of the Invention

### Problems that the Invention is to Solve

The technology of in situ depot preparations using a biodegradable polymer such as PLGA can also be applied to sustained release preparations such as hydrophilic drugs. But sometimes the problem known as “initial burst” occurs, in which immediately after application to the body the water in the body quickly infiltrates into the polymer composition, causing the drug in the composition to be released into the body quickly. Using a composition in the form of a gel tends to make it possible to reduce the initial burst as compared with an in situ depot preparation, but with a gel as disclosed in patent reference 4 that makes use of PLGA as a matrix, long-term sustained release lasting several days to several months cannot be expected.

And because a PLGA solution and gel are formed, it will be necessary to use an organic solvent such as N-methyl pyrrolidone which is highly toxic to the body. This has created the need to develop sustained release technology that can apply a solvent that is safer in the body, such as alcohol or water.

### Means of Solving the Problems

The present invention was arrived at when, as a result of study in the light of the foregoing, the inventors of the present invention found that certain amphipathic polymers, in addition to being able to form an organogel (alcogel) in which the dispersion medium is alcohol, can form a hydrogel, in which the dispersion medium is water, and that these gels can be applied as sustained release preparations that can suppress the initial burst of drugs and the like.

The present invention concerns a gel composition, and its manufacturing method, that includes an amphipathic block polymer that has a hydrophilic block chain that has 20 or more sarcosine units and a hydrophobic block chain that has 10 or more lactic acid units.

The gel composition may be either an organogel, which includes an organic solvent as the dispersion medium, a hydrogel, which includes water as the dispersion medium, or a xerogel, in which the dispersion medium has been removed. It is preferable that the gel composition of the present invention contain the above amphipathic block polymer to a content of at least 10% by weight [wt %].

An organogel composition is obtained by mixing the above amphipathic block polymer and an organic solvent. In one form, an organogel is obtained by carrying out a step in which an amphipathic block polymer is dissolved or expanded in an organic solvent under heating to make a viscous liquid that has fluidity, and a step in which the viscous liquid is cooled.

A xerogel composition is obtained by removing the organic solvent from an organogel composition. A hydrogel composition is obtained by taking a xerogel composition and wetting it with water or an aqueous solution.

The gel composition of the present invention may include a drug. A water-soluble drug may be used as the drug. An organogel composition that contains a drug is obtained by, for example, dissolving an amphipathic block polymer and a drug in an organic solvent to make a viscous liquid, and cooling the viscous liquid. An organogel composition that includes a drug is also obtained by a method in which an amphipathic block polymer is dissolved in an organic solvent to make a viscous liquid, then a drug is added to the viscous liquid, following which the viscous liquid is cooled. A hydrogel that includes a drug is obtained by making a xerogel from an organogel that includes the drug, and adding water to the xerogel that contains the drug. Also, a hydrogel that includes a drug can be made by adding water to a composition in which the drug is added to a xerogel.

### Effects of the Invention

For the gel composition of the present invention, a dispersion medium such as alcohol or water that is highly safe in the body can be used, and the initial burst of the drug or the like can be suppressed, for excellent slow release of the drug. Thus the gel composition of the present invention can be applied to a sustained release preparation for the purpose of applying it to the body.

### BRIEF EXPLANATION OF THE DRAWINGS

FIG. 1 Photographs of organogels in which the dispersion medium is (A) methanol, (B) ethanol, and (C) 2-butanol.

FIG. 2 TEM view of an organogel in which the dispersion medium is methanol.

FIG. 3 TEM view of an organogel in which the dispersion medium is ethanol.

FIG. 4 TEM view of an organogel in which the dispersion medium is 2-butanol.

FIG. 5 Graph showing the results of an organogel sustained release property test.

FIG. 6A Photograph of a xerogel after removal of the dispersion medium from an organogel.

FIG. 6B Photograph of a hydrogel in which a xerogel is wetted by distilled water.

FIG. 7 Graph showing the results of a hydrogel sustained release property test.

FIG. 8 Graph showing the results of a irritativeness test using a cornea model.

FIG. 9 Sensagram of an adsorption test of a gel composition onto mucin.

FIG. 10 Sensagram of a separation test of a gel composition.

FIG. 11 Graph showing the difference between the sensagram of a separation test from a gold surface and the sensagram of a separation test from mucin.

### EMBODIMENTS OF THE INVENTION

The gel composition of the present invention includes an amphipathic block polymer that has a hydrophilic block chain and a hydrophobic block chain. The gel composition may be in the form of an organogel, which includes an organic solvent as the dispersion medium, a hydrogel, which includes water as the dispersion medium, or a xerogel, in which the dispersion medium has been removed.

[Amphipathic Block Polymer]

The gel composition of the present invention is a composition whose main constituent element is an amphipathic block polymer that has a hydrophilic block chain and a hydrophobic block chain. The hydrophilic block chain of the amphipathic block polymer has sarcosine units as the monomer units, and the hydrophobic block chain has lactic acid units as the monomer units.

(Hydrophobic Block Chain)

The hydrophobic block includes 10 or more lactic acid units. Polylactic acid has excellent body compatibility and safety. And from the fact that polylactic acid has excellent biodegradability, its metabolism is rapid, and its accumulation in the body is low. Thus an amphipathic polymer having constituent blocks of polylactic acid is useful for biological applications, especially in the human body. And because polylactic acid is crystalline, even if the hydrophobic block chain is short, hydrophobic block chains easily coagulate, and a physical gel readily forms, in a solvent such as alcohol. Thus drug and other compounds are readily taken into a physical gel, and a polymer matrix that has a slow-release property can be formed.

There are no particular restrictions on the upper limit of the number of lactic acid units in the hydrophobic block chain, but from the viewpoint of having a stable structure, it should be no more than 1000 units. The number of lactic acid units in a hydrophobic block is preferably 10 to 1000 units, more preferably 15 to 500 units, and even more preferably 20 to 100 units.

The lactic acid units that make up the hydrophobic block chain may be either L-lactic acid or D-lactic acid. Or L-lactic acid and D-lactic acid may both be present. In a hydrophobic block chain, the lactic acid units may be all continuous, or may be discontinuous. There are no particular restrictions on the monomer units other than lactic acid that are included in the hydrophobic block chain, but we can list hydrophobic amino acids or amino acid derivatives such as, for example, glycolic acid, hydroxy acids such as hydroxy isobutyric acid, and hydrophobic amino acids such as glycine, alanine, valine, leucine, isoleucine, proline, methionine, tyrosine, tryptophan, methyl ester glutamate, benzyl ester glutamate, methyl ester aspartate, and benzyl ester aspartate.

(Hydrophilic Block Chain)

The hydrophilic block chain includes 20 or more sarcosine units (N-methyl glycine units). Sarcosine is highly water soluble. And polysarcosine, having N-substituted amide, can be cis-trans isomerized, and is very flexible because there is little steric hindrance around the a carbon. Thus, by using polysarcosine chains as constituent units, a hydrophilic block chain is formed that is both highly hydrophilic and flexible.

If there are 20 or more sarcosine units in a hydrophilic block chain, then hydrophilic blocks in any neighboring block polymer easily coagulate together, so a gel is readily formed that takes in a hydrophilic dispersion medium such as water or alcohol, as well as hydrophilic drugs and the like. There are no particular restrictions on the upper limit of the number of sarcosine units in a hydrophilic block chain. From the viewpoint of causing hydrophobic blocks of amphipathic polymers of any neighboring block polymers to coagulate together and stabilizing the structure of the gel, the number of sarcosine units in a hydrophilic block chain should be no more than 300. The number of sarcosine units is more preferably 20 to 200 units, and even more preferably 30 to 100 units.

The sarcosine units in a hydrophilic block chain may be all continuous, or, as long as the properties of the polysarcosine are not damaged, the sarcosine units may be discontinuous. If a hydrophilic block chain has monomer units other than sarcosine, there are no particular restrictions on the monomer units other than sarcosine, but one may list, for example, hydrophilic amino acids or amino acid derivatives. The amino acids include a amino acids, β amino acids, and γ amino acids, with α amino acids being preferable. As hydrophilic α amino acids we may list serine, threonine, lysine, aspartic acid, glutamic acid, etc. And a hydrophilic block may have a glycan or polyether, etc. A hydrophilic block should have a hydrophilic base, such as a hydroxyl group, at its end (the end on the opposite side from the linker part with the hydrophilic block).

(Structure and Synthesis Method of the Amphipathic Block Polymer)

In an amphipathic polymer a hydrophilic block chain and a hydrophobic block chain are joined together. The hydrophilic block chain and the hydrophobic block chain may be joined together via a linker. As the linker, it is preferable to use something which has a functional group (for example, a hydroxyl group, amino group, etc.) that can be joined to a lactic acid monomer (lactic acid or lactide) or a polylactic acid chain, which are constituent units of the hydrophobic block chain, and has a functional group (for example, an amino group) that can be joined to sarcosine or a sarcosine monomer (for example, sarcosine or N-carboxyl anhydride), which are constituent units of the hydrophilic block. Judicious selection of the linker makes it possible to control the branching structure of the hydrophilic block chain or the hydrophobic block chain.

There are no particular restrictions on how amphipathic block polymers are joined together; one may use any publicly known peptide linking methods, polyester linking methods, depsipeptide linking methods, etc. For the details, see WO 2009/148121 (the above patent reference 2); amphipathic block polymers can be synthesized.

In order to regulate the gel stability and the biodegradability of the gel and its release behavior for drugs, etc., it is desirable to regulate the polylactic acid chain length on the hydrophobic block chain and the ratio of the chain lengths of the hydrophobic block chain and the hydrophilic block chain (the ratio of the number of lactic acid units to the number of sarcosine units). In order to easily control the polylactic acid chain length, when synthesizing the amphipathic block polymer, it is preferable to first synthesize polylactic acid on one end of which a linker is introduced, then introduce polysarcosine. The chain lengths of the polysarcosine chain and the polylactic acid chain can be adjusted by adjusting the conditions such as the charging ratios of the initiators and monomers in the polymerization reactions, the reaction time, and the temperature. The chain lengths of the hydrophilic block chain and the hydrophobic block chain (the molecular weight of the amphipathic block polymers) can be confirmed by, for example, ¹H-NMR. For increasing the biodegradability of the amphipathic polymer, the weight average molecular weight should be no greater than 10000, and preferably no greater than 9000. In the amphipathic polymers used in the present invention, chemical crosslinks may be formed between molecules for such purposes as promoting gel formation and improving the stability of the gel.

[Gel Composition]

<Organogels>

An organogel is obtained by mixing the above amphipathic polymer with an organic solvent. Preferable as organic solvents for forming an organogel are solvents in which it is easy to dissolve the hydrophilic block chain and difficult to dissolve the hydrophobic block chain of the amphipathic polymer. Specifically, it is preferable to use an organic solvent that dissolves polysarcosine and does not dissolve polylactic acid. By using such an organic solvent, during mixing of the amphipathic polymer and the organic solvent, the hydrophobic block part of the amphipathic polymer coagulates, making it easy for a matrix that is physically crosslinked to form. And if an organogel is formed using such an organic solvent, the xerogel resulting after removing the organic solvent will also readily take on a structure in which the hydrophobic block part coagulates. Thus, it is thought, when water or an aqueous solution is brought into contact with the xerogel, water will readily infiltrate into the hydrophilic block chain part, and a hydrogel will readily form that retains the same polymatrix structure as the organogel.

Preferable as the organic solvent be used for forming an organogel is alcohol with 1 to 6 carbon atoms. Preferable among these is alcohol with 1 to 4 carbon atoms, because a hydrophilic block chain is highly soluble in it, and it is easy to form a xerogel by removing the organic solvent. As preferable organic solvents we can list specifically methanol, ethanol, propanole, 2-propanol, butanol, 2-butanol, etc.

Two or more organic solvents may be used mixed together. The solubility of the hydrophobic block chain and hydrophilic block chain may be adjusted by mixing together two or more organic solvents. And by adding an organic solvent of low solubility with respect to the hydrophobic block chain once the amphipathic polymer has been dissolved using the high-solubility organic solvent, it is possible to promote physical crosslinking by coagulation of the hydrophobic block and to form a gel matrix. If two or more organic solvents are used, at least one of them should be an above-listed alcohol. Two or more alcohols may be used. If the organic solvent is a mixed solvent of two or more organic solvents, at least 50 wt % of the total quantity of organic solvent should be of the above-listed alcohols. It is more preferable that the quantity of alcohol with respect to the total quantity of organic solvent be 60 wt % or more, and even more preferable that it be 70 wt % or more.

There are no particular restrictions on the ratio of amphipathic polymer to organic solvent; it may be set within the range in which the amphipathic polymer can dissolve and expand, in accordance with the molecular weight of the amphipathic polymer and the type of organic solvent, etc. From the viewpoint of keeping neighboring amphipathic polymers at a suitable distance and controlling the formation of the gel, the quantity of organic solvent is preferably 100 to 1500 parts by weight, and more preferably 200 to 1000 parts by weight. The content of amphipathic block polymer in an organogel composition should be 10 wt % or more.

In the formation of an organogel, a method is preferably adopted in which by having both an amphipathic polymer and an organic solvent present during heating, the amphipathic block polymer is made to dissolve or swell in the organic solvent, making a viscous liquid that has fluidity, then the viscous liquid is cooled. The heating makes the molecular motion of the polymer more active, thereby promoting the swelling or dissolution of the amphipathic polymer by the organic solvent. When the product of the dissolution or swelling of the amphipathic block polymer is cooled to the gel point or below, the formation of physical crosslinks of the hydrophobic block chain is promoted, resulting in an organogel of low fluidity (or of no fluidity at all).

<Xerogels>

A xerogel (dry gel) is obtained by removing the organic solvent, which serves as a dispersion medium, from an organogel. There are no particular restrictions on how the organic solvent is to be removed from the organogel; this includes the method of precipitating the gel by contact with a non-solvent, as well as drying by a gas such as nitrogen, air drying, drying by heating, heat vacuum drying, freeze drying, and supercritical drying, etc. The solvent may be removed after pulverizing the organogel and forming it into particles, for such purposes as promoting removal of the organic solvent. Also, the gel may be pulverized while removing the solvent.

There are no particular restrictions on the extent of removal of the organic solvent, but it is preferable that enough of the solvent be removed so as to result in a solid form having no dampness. The quantity of dispersion medium in the xerogel is preferably 20 wt % or less with respect to the total quantity of the gel composition, more preferably 10 wt % or less, and even more preferably 5 wt % or less. When forming a xerogel from an organogel, by removing enough of the organic solvent, the quantity of organic solvent in the hydrogel that is formed from the xerogel can be reduced, making it safer for the body.

<Hydrogels>

A hydrogel is obtained by bringing an organogel or xerogel into contact with water or an aqueous solution. A method in which a xerogel is wetted by water or an aqueous solution is preferable, because it facilitates the formation of a hydrogel and can reduce the remaining organic solvent. As an aqueous solution for forming a hydrogel, it is preferable to use an aqueous solution that is biochemically and pharmacologically permissible, such as distilled water for injection, saline solution, or a buffering solution. One can also make a hydrogel by administering an organogel or xerogel to the body, and wetting the gel with the moisture content inside the body.

There are no particular restrictions on the ratio of amphipathic polymer to water; this can be set within the range that allows wetting of the gel, in accordance with the molecular weight and mass and other properties of the amphipathic polymer. And if a hydrogel is introduced into the body by injection, the quantity of water may be adjusted so that the hydrogel is within the viscosity range that allowed injection of the hydrogel. From the viewpoint of keeping a suitable distance between the molecules of adjacent amphipathic block polymers and maintaining the strength of the gel, the quantity of water in the hydrogel is preferably 50 to 1500 parts by weight for every 100 parts by weight of the amphipathic polymer, and more preferably 100 to 1000 parts by weight. The quantity of amphipathic block polymer in the hydrogel composition should be 10 wt % or more.

A xerogel may be formed by removing the water after formation of a hydrogel. For example, if the gel composition contains a drug that is insoluble in an organic solvent or a drug that is easily broken down by an organic solvent, then a drug-containing xerogel can be obtained by mixing these drugs in a hydrogel, then removing the water. The resulting xerogel may be either applied as is, or used as a hydrogel by wetting it again with water or an aqueous solution.

From the viewpoint of reducing toxicity or irritability to the body, a hydrogel should contain as little organic solvent as possible. The proportion of water in the total dispersion medium of the hydrogel is preferably 80 wt % or more, more preferably 90 wt % or more, even more preferably 95 wt % or more, and especially preferably 98 wt % or more. In order to reduce the content of organic solvent, it is preferable to increase the rate of removal of organic solvent when the xerogel is formed from the organogel. The content of organic solvent can also be reduced by forming a hydrogel and forming a xerogel by removal of the dispersion medium.

<Other Components Making Up the Composition>

The gel composition of the present invention may contain components other than the above amphipathic polymer and dispersion medium. For example, a drug may be included in the gel composition. There are no particular restrictions on the drug, as long as it affects the body and is physiologically permissible; one may include an anti-inflammatory drug, a painkilling drug, an antibiotic, a cell cycle inhibitor, a local anesthetic, a vascular endothelial growth factor, an immunosuppressive drug, a chemotherapy drug, a steroidal drug, a hormone drug, a growth factor, a psychotropic drug, an anti-cancer drug, an angiogenesis drug, an angiogenesis inhibitor, an antiviral drug, a protein (enzyme, antibody, etc.), a nucleic acid, etc. As the drug, various ophthalmic remedies may be included. Specific examples of ophthalmic remedies include brinzolamide, povidone-iodine, betaxolol hydrochloride, ciprofloxacin hydrochloride, natamycin, nepafenac, travoprost, fluorometholone, bimatoprost, prednisolone acetate, dipivefrine hydrochloride, cyclosporin, loteprednol etabonate, pegaptanib sodium, azelastine hydrochloride, latanoprost, timolol, etc.

There are no particular restrictions on how to put the drug into the gel composition; a drug may be added to an organogel or hydrogel and be mixed in. In order to obtain a gel with an excellent drug sustained release property, it is preferable that the drug be present in the system since before the gel is formed. In particular, in a case in which a water-soluble drug is to be put into the gel composition, if the drug is present in the system before gel formation, then when the polymatrix is formed by physical crosslinks of the hydrophobic block part, the drug, together with the dispersion medium, will readily be taken into the hydrophilic part, which is present dispersed in the polymer matrix, thereby improving the sustained release property, it is inferred.

For example, if an organogel is to be formed by a method in which a viscous liquid having fluidity is made by dissolving or wetting an amphipathic block polymer in an organic solvent and then the viscous liquid is cooled, it is preferable that the drug be included in the system beginning with a stage prior to cooling the viscous liquid. As to how to get the drug into the system before the viscous liquid is cooled, we may cite methods in which the amphipathic block polymer and the drug are both dissolved in the organic solvent, methods in which an organic solvent in which the drug has previously been dissolved is mixed with the amphipathic block polymer, and methods in which a viscous liquid having fluidity is made by dissolving or wetting the amphipathic block polymer in an organic solvent, then the drug is added to the viscous liquid. Even among these, from the viewpoint of having the drug uniformly present in the gel composition, a method in which the amphipathic block polymer and the drug are both dissolved in the organic solvent is particularly preferable.

By removing the solvent from an organogel that contains a drug, a xerogel is obtained that includes the drug in the polymer matrix. By wetting this xerogel with water or an aqueous solution, a hydrogel is obtained that includes the drug. And a hydrogel that contains a drug can be made by adding water to a composition in which a drug is added to a xerogel.

The gel composition may include additional components other than a drug. Such additional components may be, for example, various solvents, preservatives, plasticizers, surfactants, antifoaming agents, stabilizers, buffering agents, pH adjustment agents, osmotic pressure regulation agents, isotonicity agents, etc. These added components may be added at any stage in the preparation of the gel composition.

[Uses for a Gel Composition]

If the gel composition of the present invention includes a drug, it can be used as a treatment gel composition to be administered to a patient. By administering the gel including the drug into the body, it can work as a sustained release preparation. It can be administered to either humans or non-human animals.

As shown below in working example 6, the gel composition of the present invention is superior in its interaction with mucin. Mucin, which is an aggregate of glycoproteins, is found everywhere on the surface of the membranes of the body. The mucous membranes of the digestive organs, nasal cavity, eyes, and elsewhere are all covered with mucin, so if the gel composition of the present invention, which has a high degree of interaction with mucin, is administered to the body, the gel composition will tend to stick to the surface of the membranes and stay there. Therefore the gel composition of the present invention is useful as a sustained release preparation that acts inside the body.

There are no particular restrictions on how to administer the gel composition to the body. As administration methods, we can list, among others, via the mucous membranes, orally, by eyedrops, percutaneously, through the nose, intramuscularly, subcutaneously, intraperitoneally, in the joints, in the areole, in the wall or lining, during an operation, in the crown of the head, in the peritoneum, in the pleura, in the lungs, in the bone marrow cavity, in the chest cavity, in the trachea, in the atrium of the ear, in the uterus, etc. The gel composition can be made with suitable properties and conditions according to the administration site and method.

For example, with an organogel or a hydrogel, if the viscosity is adjusted suitably, it can be made to act as a depot preparation administered to the body by a subcutaneous injection. And because an organogel or a hydrogel can be administered by spreading it on, it is also suitable for forms such as percutaneous administration or administration through a mucous membrane.

As compared with conventional in situ gelled depot preparations, the organogel composition of the present invention can to a greater degree suppress the initial burst of the drug and maintain the sustained release property longer. And it can enhance bodily safety because it can use alcohol as a dispersion medium, which is less toxic to the body than N-methyl pyrrolidone or the like. With the hydrogel composition of the present invention the initial burst of drug is suppressed, and bodily safety is improved even beyond that of an organogel. In particular, it causes almost no irritation to the cornea, which makes it suitable as a sustained release drug such as an ophthalmic eyedrop drug.

With the gel composition of the present invention, it is preferable during storage to store it as a xerogel composition having no dispersion medium, and to make an organogel or hydrogel, etc. by adding a dispersion medium immediately before it is applied to the body. By storing the gel preparation with no dispersion medium being present, any hydrolysis or the like of the amphipathic polymer in the storage environment is suppressed, and the sustained release property of the drug when administered to the body can be maintained at a high level.

Because the gel composition of the present invention has a drug sustained release property, one can also expect its application as a carrier in a drug delivery system (DDS). And by including in the gel composition a fluorescent marker or other signaling preparation as a drug, one can also expect its practical application as a probe for living-body imaging, such as fluorescent imaging, ultrasonic imaging, or optical-acoustic imaging. Even if the gel composition does not include a drug, the gel composition can be used as a filler or the like. Applications for the gel composition of the present invention can also be expected in fields other than medical uses, such as cosmetics, foods, and agriculture.

### WORKING EXAMPLES

In the following we describe the present invention in greater detail by presenting working examples, but the present invention is not limited to these examples.

### Synthesis Example

### Synthesis of an Amphipathic Block Polymer

Referring to the method described in WO 2009/148121, with sarcosine anhydride and aminated poly L-lactic acid as monomer components, using glycolic acid, O-(benzotriazole-1-yl)-N,N,N′,N′-tetra methyl uronium hexafluoro phosphate (HATU), and N-diisopropyl ethyl amine (DIEA), a straight-chain amphipathic block polymer (PLA₃₀-PSar₇₈) was synthesized that has a hydrophilic block made up of 78 sarcosine units and a hydrophobic block made up of 30 L-lactic acid units.

### Working Example 1

### Preparing an Organogel

### Preparation Example 1A

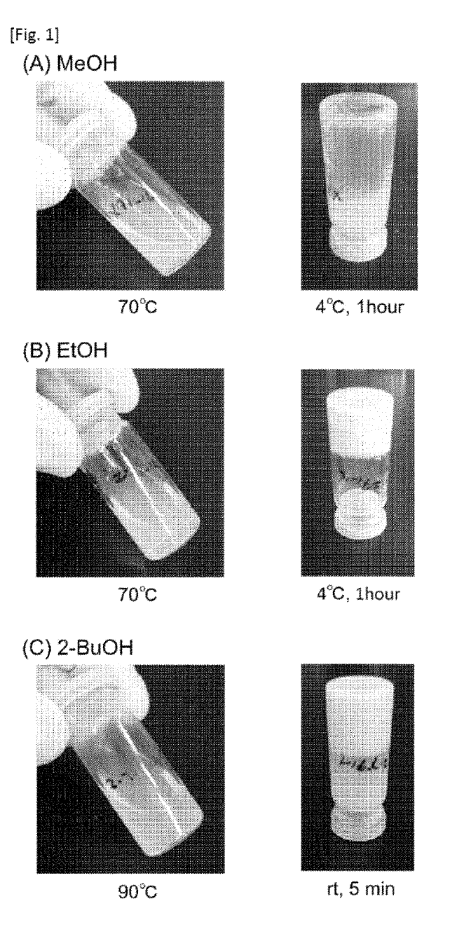

To 500 mg of the polymer obtained in the synthesis example was added 2.5 mL of methanol (MeOH), and when heated to 70° C., the polymer broke down and a milky-white solution was obtained (FIG. 1(A) on the left). This solution was cooled at 4° C. for 1 hour, resulting in a fluid gel having viscosity (FIG. 1(A) on the right).

### Preparation Example 1B

To 500 mg of the polymer obtained in the synthesis example was added 2.5 mL of ethanol (EtOH), and when heated to 70° C., the polymer broke down and a milky-white solution was obtained (FIG. 1(B) on the left). This solution was cooled at 4° C. for 1 hour, resulting in a white wet gel not having fluidity (FIG. 1(B) on the right).

### Preparation Example 1C

To 500 mg of the polymer obtained in the synthesis example was added 2.5 mL of 2-butanol (2-BuOH), and when heated to 90° C., the polymer broke down and a yellow-tinged milky-white solution was obtained (FIG. 1(C) on the left). This solution was cooled at room temperature for 5 minutes, resulting in a white wet gel not having fluidity (FIG. 1(C) on the right).

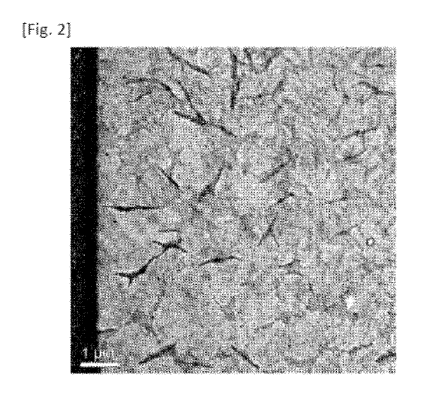

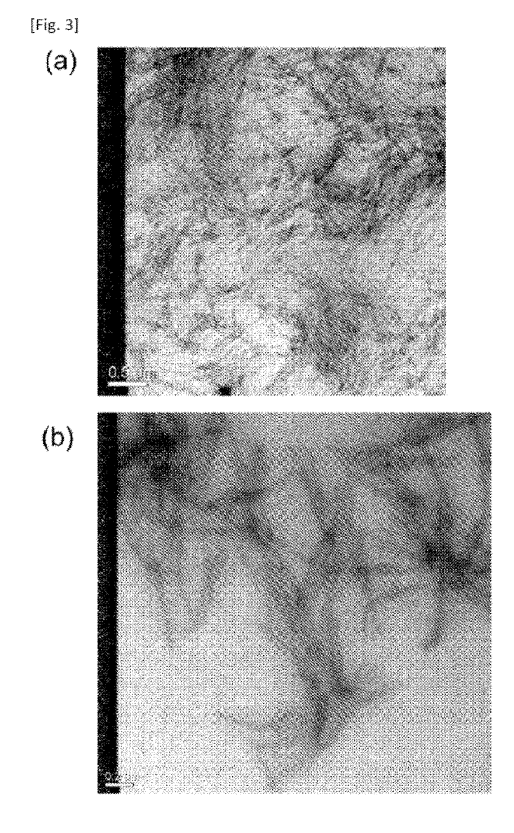

In order to confirm the fine structure of the gels obtained in the above preparation examples 1A to 1C, an observation was conducted with a transmission electron microscope (TEM). FIG. 2 is the TEM observation image of the gel (preparation example 1A) in which methanol was used. FIG. 3 is the TEM observation image of the gel (preparation example 1B) in which ethanol was used; (a) is a low-magnification image, and (b) is a high-magnification image. As shown in FIG. 2 and FIG. 3, it was confirmed that in the gel in which methanol and ethanol were used, the structure was one in which fibrous structures with a width of several tens of nm and a length of about 1 μm lined up.

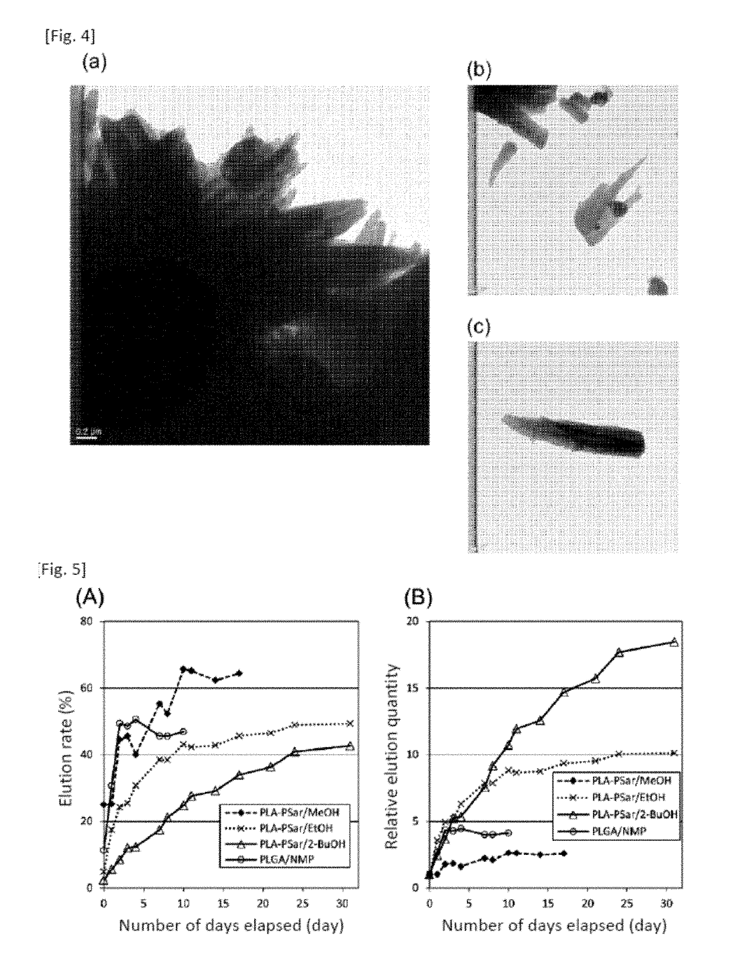

FIG. 4 is the TEM observation image of the gel (preparation example 1C) in which 2-butanol was used. As shown in FIG. 4(a), with the gel in which 2-butanol was used, a gel was formed in which rod-shaped structures coagulated together. FIGS. 4(b) and (c) are TEM observation images of free structures, and rod-shaped structures were confirmed having a width of several hundred nm and a length of several μm.

### Working Example 2

### Organogel Drug Sustained Release Property Test

Preparation of the Samples

### Preparation Example 2A

In the same way as in preparation example 1A, polymer was dissolved in methanol, 2.5 mg of fluorescein isothiocyanate marker dextran (FITC-dextran) was added, then cooling was carried out, resulting in an organogel having fluidity.

### Preparation Example 2B

In the same way as in preparation example 1B, polymer was dissolved in ethanol, 2.5 mg of FITC-dextran was added, then cooling was carried out, resulting in an organogel not having fluidity.

### Preparation Example 2C

In the same way as in preparation example 1C, polymer was dissolved in 2-butanol, 2.5 mg of FITC-dextran was added, then it was left to cool at room temperature, resulting in an organogel not having fluidity.

### Preparation Example 2D

### Preparation of Solution Using PLGA (Comparison Example)

To 500 mg of PLGA (random copolymer of L-lactic acid and glycolic acid with mole ratio 1:1) of weight average molecular weight about 5000 was added 611 mg of N-methyl pyrrolidone (NMP), and after dissolution, 2.5 mg of FITC-dextran was added to obtain a solution.

### Preparation Example 2E

### Preparation of Polymer Micelle-Containing Composition

### Comparison Example

The polymer obtained in the synthesis example was dissolved in chloroform to obtain a 2 mg/mL polymer solution. This polymer solution was put into a glass test tube, and by evaporating the solvent under reduced pressure using an evaporator, a polymer film was formed on the wall surface of the test tube. In addition, after carrying out all-night vacuum drying at room temperature, distilled water was added to the test tube, heat treatment was carried out for 20 minutes at a temperature of 85° C., and in distilled water, nanoparticles (average particle diameter: 35 nm) were deposited consisting of amphipathic polymer micelles. The resulting dispersion liquid was freeze-dried, yielding a white powder of nanoparticles. To 500 mg of these nanoparticles was added 2.5 mg of FITC-dextran to obtain a mixture of polymer micelles and FITC-dextran.

<Sustained Release Property Test>

To each of the compositions obtained in the above preparation examples 2A to 2E, 10 mL of distilled water was added, and the container was shaken lightly. In order to determine the amount of elution of FITC-dextran from each sample into the distilled water, an aqueous solution of the clear top was sampled with a micropipette and diluted 50-fold, the fluorescence spectrum was measured, and the fluorescent intensity at a wavelength of 521 nm was determined. As a reference sample, a solution was prepared in which 2.5 mg of FITC-dextran was dissolved in 10 mL of distilled water, and the fluorescent intensity at a wavelength of 521 nm was determined from the fluorescence spectrum. The ratio of the fluorescent intensity of each sample to the fluorescent intensity of the reference sample was taken as the elution rate (%).

Each sample and the reference sample were left standing at room temperature, and each day the clear top of each sample was sampled, fluorometry was carried out, and the elution rate with respect to the reference sample was determined. FIG. 5(A) shows how the elution rate changed from day to day. FIG. 5(B) shows the day-by-day changes in the elution quantity, taking as 1 the elution rate immediately after adding the distilled water (after 0 days).

For the polymer micelle-containing composition of preparation example 2E, the elution rate at day 0 was 89%, and thereafter as well, no change was seen in the elution rate (data not shown in the drawings). From this result it is clear for the amphipathic polymer micelles that the occlusion property of FITC-dextran is poor, that substantially all of the FITC-dextran in the composition dissolves away, and that sustained release from the polymer micelles cannot be expected.

From the results shown in FIG. 5(A) it is clear that in preparation example 2D (PLGA/NMP), in which PLGA was used as the polymer matrix, the elution rate increases to about 50% after 2 days, and thereafter no increase in elution rate is seen and the elution rate is saturated. In comparison to this, with the gel composition of the synthesis example, in which the matrix is made of amphipathic block polymer, an increase in the elution rate was seen up to day 10 with the preparation example 2A (PLA-PSar/MeOH), in which the solvent was methanol, up to day 25 with the preparation example 2B (PLS-PSar/EtOH), in which the solvent was ethanol, and up to day 31 with the preparation example 2C (PLA-PSar/2-BuOH), in which the solvent was 2-butanol. Also, each of the preparation examples 2A to 2C showed a higher elution rate at saturation than did preparation example 2D.

From the results shown in FIG. 5(B), it is clear that whereas with the PLGA/NMP solution of preparation example 2D the saturated release quantity was about 4 times the release quantity on the initial day, with the ethanol gel of preparation example 2B the saturated release quantity is about 10 times the release quantity on the initial day, and with the 2-butanol gel of preparation example 2C the saturated release quantity is about 18 times the release quantity on the initial day, having an excellent sustained release property.

### Working Example 3

### Preparing a Hydrogel

### Preparation Examples 3A to 3C

When an organogel prepared under the same conditions as in the above preparation examples 1A to 1C was put in a desiccator and dried under reduced pressure overnight (for about 12 hours), a dry gel (xerogel) from which the solvent had been removed was obtained (FIG. 6A). When 2.5 mL of distilled water was added to each xerogel and it was left standing for 4 hours at room temperature, the gel was wet and a hydrogel was obtained (FIG. 6B).

### Preparation Example 3D

### Comparison Example

A solution was prepared in which 611 mg of N-methyl pyrrolidone (NMP) was added as a solvent to 500 mg of PLGA (random copolymer of L-lactic acid and glycolic acid of mole ratio 3:1) of weight average molecular weight about 20000. This solution was put in a desiccator, reduced-pressure drying was done overnight, then when 2.5 mL of distilled water was added, the polymer solidified and a hydrogel was not obtained.

### Working Example 4

### Hydrogel Drug Sustained Release Property Test

<Preparation of the Samples>

### Preparation Examples 4A to 4C

A xerogel was prepared under the same condition as in the above preparation examples 3A to 3C, and 2.5 mL of distilled water in which 2.5 mg of FITC-dextran was dissolved was added, producing a hydrogel containing FITC-dextran.

### Preparation Example 4D

### Comparison Example

After preparing a PLGA/MNP solution under the same conditions as in preparation example 3D above, 2.5 mg of FITC-dextran was added to yield a solution.

(Sustained Release Property Test)

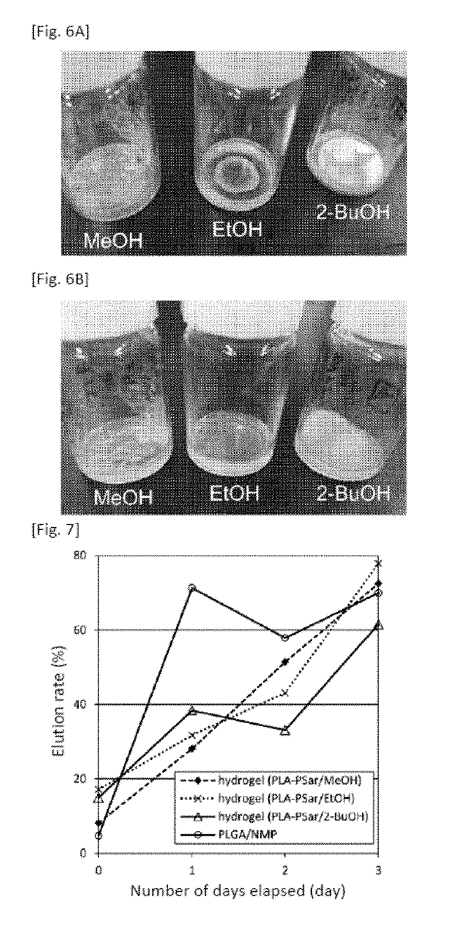

The same sustained release property test as in working example 2 was performed on samples of the FITC-dextran-containing hydrogels obtained in the above preparation examples 4A to 4C, and on the FITC-dextran-containing PLGA solution obtained in preparation example 4D. The day-to-day change in elution rate is shown in FIG. 7.

From the results shown in FIG. 7, it is clear that the sustained release property is excellent: with PLGA, whereas the elution rate exceeded 70% on day 1, with the hydrogels of preparation examples 4A to 4C obtained by drying an organogel and wetting it with water, in every one of them the elution rate increased until day 3.

### Working Example 5

### Irritativeness Test Using a Cornea Model

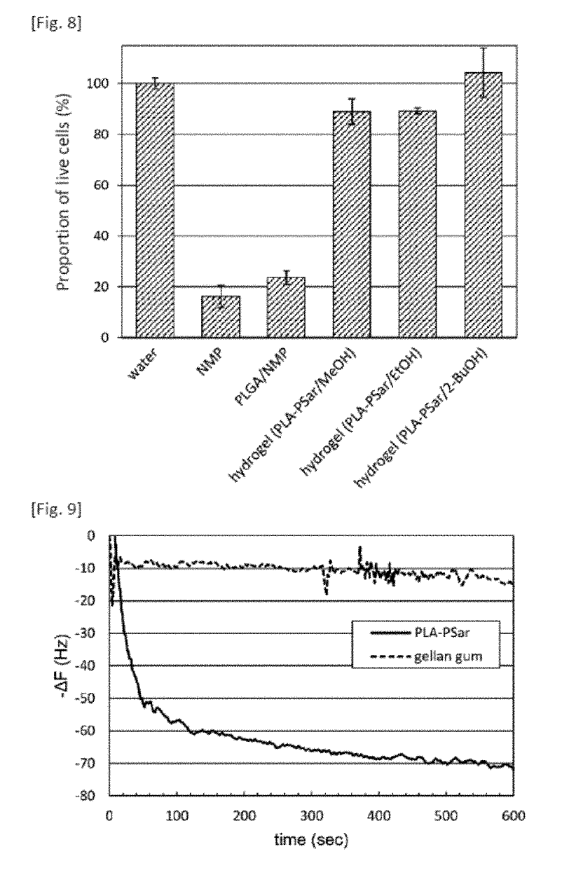

Prepared as test substances were hydrogels made under the same conditions as in the above preparation examples 3A to 3C (prepared from methanol gel, ethanol gel, and 2-butanol gel, respectively); solution in which 611 mg of NMP was added to 500 mg of PLGA; NMP; and distilled water (negative reference). A standard protocol was followed using a three-dimensional cultured corneal epithelium model from human normal corneal epithelium cells (J-TEC, LabCyte CORNEA-MODEL), and exposure tests were carried out on 50 μL of the test substances. Using a WST-8 assay kit (Dojin Chemical, product code: CK04), following the exposure test, a WST-8 assay was done on the samples, the OD value was measured with a plate reader (TECAN, Infinite 200 Pro), and the relative survival rate (proportion of live cells) was calculated for the negative object (distilled water). The results are presented in FIG. 8.

From the results shown in FIG. 8, it is clear that with the NMP solution of PLGA the proportion of live cells is about 20%, and that the irritativeness toward the cornea is strong, the same as with NMP, which is the solvent. On the other hand, the amphipathic polymer hydrogels (prepared from methanol gel, ethanol gel, and 2-butanol gel, respectively) all showed a high proportion of live cells.

From the results of working example 4 and working example 5, it is clear that hydrogels in which the matrix is made of amphipathic polymer have a superior sustained release property, their bodily irritativeness is low, and they are materials that are well suited for slow release preparations to be applied to the body.

### Working Example 6

### Confirmation of Interaction with Mucin

Using hydrogels made under the same conditions as in preparation example 3B above (prepared from ethanol gel, polymer concentration 100 mg/mL), the interaction between mucin and the gel was confirmed from changes in weight according to the QCM-A method. Used for the comparison were hydrogels of a gellan gum base (polymer concentration 100 mg/mL). Also, gellan gum is a polysaccharide that has the property of gelling and building up on the surface of the eyeball; it is a component that is used in sustained-release eyedrop medications and the like.

<Preparation of Cells to be Measured>

(Preparation of Mucin-Bonding Sensor Cells)

A QCM sensor cell equipped with a gold electrode was mounted on a QCM device, and after the start of monitoring by a sensagram, 500 μL of phosphate-buffered saline (PBS) was added into the cell. A cell cover having an attached stirrer was put on, and after the sensagram stabilized, 5 μL of 10 mg/mL mucin solution diluted with PBS was added (final mucin concentration: 100 μg/mL). After confirming the increase in weight (the bonding of the mucin to the gold surface) with the sensagram, the cell was removed from the QCM device, the PBS was discarded, and the interior of the cell was washed multiple times with distilled water.

(Preparation of a Reference Cell)

After adding 500 μL of PBS into a QCM sensor cell and stirring, the PBS was discarded without adding any mucin solution, and the interior of the cell was washed multiple times with distilled water.

### Working Example 6A

### Adsorption Test onto Mucin

A mucin bonding sensor cell was mounted on a QCM device, 500 μL of PBS was added into the cell, then monitoring began by a sensagram. To the PBS was added 10 μL of hydrogel, and its adsorption onto mucin was monitored.

### Working Example 6B

### Test of Separation from Mucin

(Monitoring of Hydrogel Adsorption)

10 μL of hydrogel was loaded onto the electrode surfaces of a mucin bonding sensor cell and the reference cell. A cell that had been loaded with gel was mounted on a QCM device, 500 μL of PBS was added into the cell, then a cell cover having an attached stirrer was put on. After the sensagram stabilized, stirring was begun, and the separation of the gel from the surface was monitored (taking the start of stirring as time 0).

<Evaluation Results>

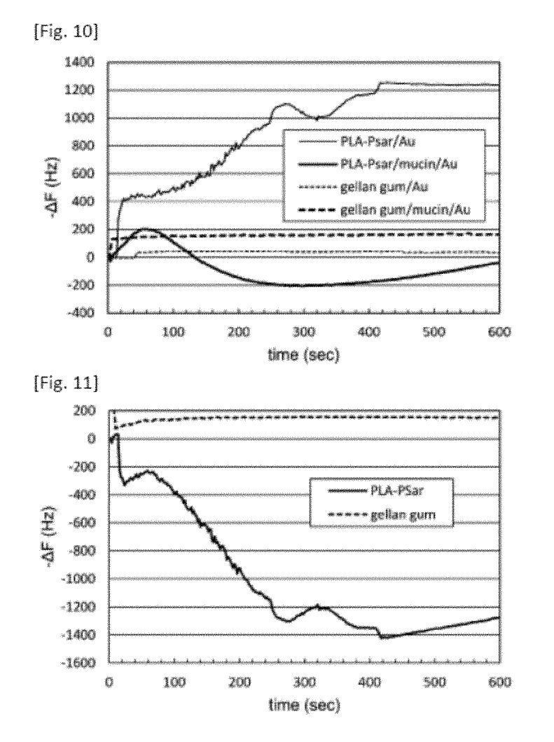

The sensagram of working example 6A (adsorption test) is shown in FIG. 9. The sensagram of working example 6B (separation test) is shown in FIG. 10A. And FIG. 11 shows the difference between the sensagram of the reference cell of working example 6A and the sensagram of the adsorption cell.

FIG. 9 makes it clear that in the gellan gum adsorption test, almost no change in the sensagram is seen, and that gellan gum adsorbs hardly at all to mucin. On the other hand, the amphipathic polymer (PLA-PSar) hydrogel showed a rapid sensagram change (weight increase) for about 50 seconds immediately after being added to the PBS, and thereafter as well, it showed a gradual change. These results make it clear that an amphipathic polymer hydrogel has high adsorption power with respect to mucin.

In FIG. 10, in the separation test of gellan gum from the gold surface, almost no change in the sensagram was seen. In the separation test of gellan gum from mucin, separation was found to a slight degree immediately after the start of stirring, but thereafter no change in the sensagram was seen. In the separation test of amphipathic polymer hydrogel from the gold surface, rapid separation of the gel was seen immediately after the start of stirring. On the other hand, in the separation test from the mucin surface, gradual separation was seen for up to about 100 seconds from the start of stirring, but thereafter a decline in the sensagram was confirmed. The decline in the sensagram (increase in weight) is thought to be because the hydrogel adsorbed onto the mucin undergoes water adsorption.

The graph in FIG. 11 shows the difference between the test that uses a mucin bonding sensor cell and the test that uses the reference cell (gold surface), indicating the bonding specificity to mucin. With gellan gum, from the fact that the separation from the gold surface and the separation from the mucin are about the same, it is thought that the interaction between gellan gum and mucin is about the same as the interaction between gellan gum and gold. On the other hand, it is clear that an amphipathic polymer hydrogel, while separating readily from a gold surface, separates slowly from mucin, and has a specific interaction with mucin.

These results make it clear that the gels of the present invention are adsorbed readily onto mucin and separate only with difficulty after adsorption due to their interaction with mucin. In other words, this suggested that if a gel of the present invention is administered in the body, the gel will adhere to the mucin that covers the surface of bodily membranes and will stick to the membrane surface. Therefore it can be said that the gels of the present invention are superior in their applicability to the body.

1. A gel composition, comprising:
an amphipathic block polymer including a hydrophilic block chain and a hydrophobic block chain,
wherein the hydrophilic block chain has 20 or more sarcosine units, and the hydrophobic block chain has 10 or more lactic acid units.

2. A gel composition of claim 1, further comprising:
a drug.

3. A gel composition of claim 2, wherein the drug is water-soluble.

4. A gel composition of claim 1, further comprising:
a dispersion medium comprising an organic solvent.

5. A gel composition of claim 4, wherein the organic solvent includes an alcohol of 1 to 6 carbon atoms.

6. A gel composition of claim 1, further comprising
a dispersion medium comprising water.

7. A gel composition of claim 4, wherein the amphipathic block polymer is included at a content of 10 wt % or more.

8. A gel composition of claim 1, further comprising:
a dispersion medium included at a content of 20 wt % or less.

9. A method of manufacturing an organogel composition, comprising:
mixing an amphipathic block polymer and an organic solvent,
wherein the amphipathic block polymer includes a hydrophilic block chain and a hydrophobic block chain, the hydrophilic block chain has 20 or more sarcosine units, and the hydrophobic block chain has 10 or more lactic acid units.

10. A method of claim 9, wherein the organic solvent includes an alcohol of 1 to 6 carbon atoms.

11. A method of claim 9, wherein the mixing includes dissolving or expanding, under heating, the amphipathic block polymer in the organic solvent such that a viscous liquid having fluidity is prepared; and
cooling the viscous liquid.

12. A method of claim 11, wherein the viscous liquid includes a drug before the cooling of the viscous liquid.

13. A method of manufacturing a xerogel composition, comprising:
preparing an organogel composition by the method of claim 9; and
removing the organic solvent from the organogel composition.

14. A method of manufacturing a xerogel composition, comprising:
removing the organic solvent from the gel composition of claim 4.

15. A method of manufacturing a hydrogel composition, comprising:
preparing a xerogel composition by the method of claim 13; and
wetting the xerogel composition by water or an aqueous solution.

16. A method of manufacturing a hydrogel composition, comprising:
wetting the gel composition of claim 8 by water or an aqueous solution.

17. A gel composition of claim 2, further comprising:
a dispersion medium comprising an organic solvent.

18. A gel composition of claim 3, further comprising:
a dispersion medium comprising an organic solvent.

19. A gel composition of claim 2, further comprising
a dispersion medium comprising water.

20. A gel composition of claim 3, further comprising
a dispersion medium comprising water.
